(19) United States
(12) Patent Application Publication
(10) Pub. No.: US 2024/0197345 A1
(43) Pub. Date: Jun. 20, 2024

(54) HOLDING DEVICE FOR A LITHOTRIPSY DEVICE FOR FRAGMENTING CALCULI, AND LITHOTRIPSY DEVICE

(71) Applicant: KARL STORZ SE & Co. KG, Tuttlingen (DE)

(72) Inventor: Bernhard GLÖGGLER, Tuttlingen (DE)

(21) Appl. No.: 18/537,613

(22) Filed: Dec. 12, 2023

(30) Foreign Application Priority Data
Dec. 15, 2022 (DE) 10 2022 133 520.8

(51) Int. Cl. A61B 17/22

(52) U.S. Cl. CPC A61B 17/22012; A61B 2017/22011; A61B 2560/0406

(57) ABSTRACT

The invention relates to a holding device for a lithotripsy device for fragmenting calculi, the holding device comprising a housing for accommodating assemblies and/or components, and a sonotrode being connectable to the distal end of the housing, arranged within the housing there being an acceleration tube with a longitudinal center axis, a cavity, and with a movable projectile within the cavity for shock excitation of the sonotrode, a proximal-side abutment element arranged at the proximal end, and a distal-side abutment element arranged at the distal end of the acceleration tube, and the holding device being assignable a force generation apparatus for generating a force for moving the projectile back and/or forth, and a vibration excitation apparatus for exciting vibrations of the sonotrode and a vibration damping apparatus being arranged in the housing, wherein the vibration damping apparatus comprises at least one mass and at least two spring elements with two ends each, the at least two spring elements contacting the mass with their respective one end and at least one spring element, with its second end, contacting an inner surface of the housing. The invention also relates to a lithotripsy device.

### CROSS-REFERENCE TO RELATED APPLICATIONS

This application claims the benefit under 35 U.S.C. 119(a) to German Patent Application No. 10 2022 133 520.8, filed 15 Dec. 2022, the disclosure of which is incorporated herein by reference in its entirety.

### BACKGROUND

The invention relates to a holding device for a lithotripsy device for fragmenting calculi, the holding device comprising a housing for accommodating assemblies and/or components, and the housing comprising a distal end and a proximal end and a sonotrode being connectable to the distal end, arranged within the housing there being, as an assembly, an acceleration tube with a longitudinal center axis, a cavity, a proximal end, and a distal end, and with a movable projectile within the cavity for shock excitation of the sonotrode, a proximal-side abutment element arranged at the proximal end, and a distal-side abutment element arranged at the distal end of the acceleration tube, and the holding device being assignable a force generation apparatus for generating a force for moving the projectile back and/or forth between the proximal-side abutment element and the distal-side abutment element, and, as an assembly, a vibration excitation apparatus for exciting vibrations of the sonotrode and a vibration damping apparatus being arranged in the housing. The invention also relates to a lithotripsy device, in particular an intracorporeal lithotripsy device, for fragmenting calculi.

Lithotripsy is a known method for fragmenting calculi which form, for example, through condensation and/or crystallization of salts and proteins in body organs, such as in the bladder or kidneys. If the calculi are too large to be passed naturally and causes discomfort, they have to be fragmented with a lithotripter so that the fragmented stones can be removed by natural excretion and/or by means of an aspiration/irrigation pump. The calculi to be subject to fragmentation are frequently structured inhomogeneously with different constituent parts and/or solidities.

To improve calculi fragmentation performance, use is made, especially in intracorporeal lithotripsy, of combination systems which combine two different excitation and/or vibration sources. To this end, intermittent, ballistic shockwave energy is frequently supplied in addition to the constant ultrasonic energy. For example, this can be implemented by means of a ballistic drive with electromagnets, in which an impact body is accelerated by means of the electromagnets and strikes on a horn and/or the sonotrode head. In pneumatic lithotripters, the projectile is instead accelerated within an acceleration tube by supplying compressed air and the kinetic energy of the projectile is transferred via an elastic shock to the proximal end of the sonotrode and onward to the distal end of the latter for fragmenting a calculus in the body.

The grip and the sonotrode of ballistic and/or pneumatic lithotripsy devices vibrate strongly, predominantly in the longitudinal direction, on account of the projectile acceleration. When the projectile accelerates in the distal direction, the pressure on the projectile also acts on the housing of the lithotripsy device at the same time, whereby the housing retreats in the opposite direction and the sonotrode tip moves away from the calculus. As a result, the sonotrode tip is no longer optimally aligned on the calculus and/or shifts laterally on account of a lack of static friction. During the opposite return movement of the projectile in the proximal direction, the housing moves oppositely and hence the sonotrode tip moves in the distal direction, giving rise to the risk of the calculus being pressed away from the sonotrode tip, being lost in the body tissue, and/or causing damage in said body tissue.

Additionally, combined lithotripsy devices see transverse moments being exerted on the sonotrode as a result of the ultrasound excitation and corresponding vibrations being applied to the housing. Hence, it is more difficult for the user to accurately align the sonotrode tip with the calculus on account of these differently aligned vibrations, and the user must repeatedly realign the sonotrode tip during the application. Moreover, the user feels uncomfortable and/or even painful vibrations on the housing and/or grip of the hand-held instrument, and these interfere with handling and operation.

Various approaches for reducing lithotripter vibrations, for example vibrationally decoupling the housing from the assemblies accommodated in the housing, frequently lead to the handpiece becoming very heavy and difficult to handle by the user. For example, a floating mount in a second, surrounding housing around the handpiece for decoupling the vibrations from the hand of the user directly increases the diameter and the weight, to the detriment of the haptics and ergonomics of the handling. Moreover, the vibrations triggered by the acceleration of the projectile in the distal direction and the distal impact of the projectile can only be decoupled with difficulty, also on account of the restricted installation space in the distal end portion of the handpiece, and a fast and/or hard distal-side impact of the projectile is desirable, especially in view of a fast fragmentation of hard stones in particular.

In the context of exciting vibrations by means of ultrasound, it is also known to arrange an ultrasonic vibration compensator in ultrasonic transducers, on the side opposite to the horn and hence at the vibrating proximal end of the ultrasonic converter, said vibration compensator serving as a mechanical fastening element between a housing of the lithotripsy device at rest and the vibrating, proximal end of the ultrasonic converter. In the case of a targeted design of this ultrasonic vibration compensator, the latter reduces the ultrasonic vibrations to a minimum or zero over its length, without the ultrasonic converter being noticeably detuned in terms of its resonant frequency in the process. However, it is not possible to design the dimensions of such an ultrasonic vibration compensator to take any desired values since, if this were the case, the ultrasonic converter would undesirably detune, undesirable transverse vibrations would be excited, and/or uncomfortable noises might occur. Moreover, it is not possible to freely design the housing length in the proximal direction.

It is an object of the invention to improve the prior art.

The object is achieved by a holding device for a lithotripsy device for fragmenting calculi, the holding device comprising a housing for accommodating assemblies and/or components, and the housing comprising a distal end and a proximal end and a sonotrode being connectable to the distal end, arranged within the housing there being, as an assembly, an acceleration tube with a longitudinal center axis, a cavity, a proximal end, and a distal end, and with a movable projectile within the cavity for shock excitation of the sonotrode, a proximal-side abutment element arranged at the proximal end, and a distal-side abutment element arranged at the distal end of the acceleration tube, and the holding device being assignable a force generation apparatus for generating a force for moving the projectile back and/or forth between the proximal-side abutment element and the distal-side abutment element, and, as an assembly, a vibration excitation apparatus for exciting vibrations of the sonotrode and a vibration damping apparatus being arranged in the housing, wherein the vibration damping apparatus comprises at least one mass and at least two spring elements with two ends each, the at least two spring elements contacting the mass with their respective one end and at least one spring element, with its second end, contacting an inner surface of the housing.

Consequently, a handpiece for a combined lithotripsy device with a shock excitation and a vibration excitation of the sonotrode is provided, in which the vibrations induced on account of the shock excitation and the vibration excitation are significantly reduced by means of the vibration damping apparatus. As a result, the handpiece rests steadier in the hand of the user and is easier to manage without the momentum transfer to the connectable or connected sonotrode being restricted. The vibration damping apparatus is used to simultaneously prevent or at least reduce undesirable excited vibrations on the acceleration tube, generated by the vibration excitation apparatus, or, conversely, on the vibration excitation apparatus, generated by the force generation apparatus as a result of a distal-side impact of the projectile, whereby the vibration excitation apparatus and the ballistic drive by means of the force generation apparatus are settable and operable independently of one another.

It is particularly advantageous that the vibration damping apparatus is integrable in space-saving fashion within the usually available installation space in the housing, for example to the proximal side of the vibration excitation apparatus, so that the installation space and the weight of the handpiece are not increased or only increased slightly. In addition to the compact arrangement of the vibration damping apparatus within the available housing, the requirements in respect of safety and cleanability of the handpiece are also fulfilled.

As a result of configuring the vibration damping apparatus with at least one mass, which acts as an inertial mass and/or absorber mass, and with at least two spring elements, which act as vibration damping means, both the intensity of the vibrations and their further transmission to the inner surface of the surrounding housing are significantly reduced and decoupled. In this case, the mass as absorber mass especially reduces the recoil following the impact of the projectile on the distal-side or proximal-side abutment element. This improves the handling of the lithotripsy device for the user and the accurate spatial positioning of the sonotrode tip on and/or in the calculus to be fragmented since, on account of the vibration damping apparatus, the sonotrode tip does not move away from the calculus to be fragmented to such a great extent when the projectile accelerates in the distal direction or, in case of the opposite acceleration direction of the projectile, the sonotrode tip does not inadvertently push the calculus away.

An essential concept of the invention is based on the use in a targeted manner of a vibration damping apparatus which is arranged in the housing of the holding device and comprises at least one mass and at least two spring elements, with at least one spring element contacting an inner surface of the housing with its one end and contacting the mass of the vibration damping apparatus with its other end, to quench, damp, and/or largely decouple from the housing vibrations generated by a vibration excitation and a shock excitation in the lithotripsy device. In this case, the vibration damping apparatus is matched in a targeted manner to a specific frequency range and/or the operating frequency of the vibration excitation apparatus, with the result that the desired vibration excitation and shock excitation of the sonotrode are not restricted. Moreover, a recoil following the distal-side impact of the projectile can also be reduced by an arrangement of the vibration damping apparatus to the proximal side of the vibration excitation apparatus.

The following concepts shall be explained:

A “lithotripsy device” (also called a “lithotripter”) is in particular a device for fragmenting calculi by shocks, shock waves, deformation waves, and/or vibration waves. A lithotripsy device is understood to include, in particular, various structural parts, constructional and/or functional components of a lithotripter The lithotripsy device can completely or partially form a lithotripter. A lithotripsy device can be in particular be an intracorporeal or extracorporeal lithotripsy device. In the case of an intracorporeal lithotripsy device, the latter can additionally comprise an irrigation/aspiration pump. The lithotripsy device can be designed as hand-held equipment and/or can comprise an endoscope or be inserted into an endoscope. The lithotripsy device is in particular autoclavable and has, for example, instrument steel and/or plastic. The lithotripsy device can comprise further components, such as control and/or supply equipment, or this is assigned to the lithotripsy device. In particular, a lithotripsy device is a combined lithotripsy device with a ballistic and/or pneumatic unit and an assignable force generation apparatus and a vibration excitation apparatus. By way of a shock energy when a projectile strikes a distal-side abutment element, a deformation wave shaped in a targeted manner is impressed directly or indirectly on the sonotrode, in particular, by means of the ballistic and/or pneumatic unit and the assignable force generation apparatus. The deformation wave causes in particular a translational movement of the sonotrode, which causes fragmentation of stones on account of the deflection. In addition to the mechanical shock, the sonotrode in the combined lithotripsy apparatus is excited to vibrate at the same time, in particular as a longitudinal vibration and/or transverse vibration, in particular by means of a vibration excitation apparatus, for example an ultrasonic transducer. Thus, the sonotrode is designed in particular as a waveguide for the vibration waves generated by the vibration excitation apparatus and for the deformation waves of the projectile.

The term “calculi” (also referred to as “concretions”) refers in particular to all stones in a human or animal body, which are formed for example from salts and proteins by crystallization and/or condensation. The calculi can include gallstones, urinary stones, kidney stones and/or salivary stones. As a result of the sonotrode and/or hollow probe acting on the calculi, calculus cores (also referred to as drilled cores) and/or calculus fragments in particular arise.

A “holding device” (also referred to as a “handpiece”) is a grip and/or holding part of the lithotripsy device in particular. The holding device can be in particular a handle for manual and/or automated operation and/or connection of the lithotripsy device. A holding device can also be arranged at, connected to and/or guided in an automated manner at a distal end of a robot arm. In particular, the holding device comprises a housing. The holding device can also be formed from two or more parts. For example, the holding device may comprise a separate housing for a pneumatic unit and a separate housing for the vibration excitation apparatus.

The terms “distal-side” and “distal” refer to an arrangement close to the patient's body and thus remote from the user, and/or a corresponding end or section. Accordingly, “proximal-side” or “proximal” refers to an arrangement close to the user and thus remote from the patient's body, or a corresponding end or section.

A “sonotrode” is in particular a component which, by the action and/or introduction of mechanical vibrations, is itself set in vibration and/or resonant vibration. In particular, the sonotrode is designed as a waveguide for the vibration waves generated by the vibration excitation apparatus and for the deformation waves resulting from the impact of the projectile is accelerated by means of the force generation apparatus. In particular, the sonotrode is directly or indirectly connected to the vibration excitation apparatus, the ultrasonic transducer, and/or the horn. For example, the sonotrode is screwed into the distal-side end of the horn. The sonotrode comprises a sonotrode head, in particular at its proximal end, for recording, transmitting, and/or focusing ultrasonic waves and a sonotrode tip, at its distal end, for directly and/or indirectly impacting on and/or contacting calculi. The sonotrode is in particular shaped in such a way that it optimally introduces the vibration waves, the ultrasonic vibration, and the deformation waves at its distal end into the body, into the region of the body to be treated, and/or directly onto the calculus to be fragmented in the body. In the case of ultrasonic excitation, the sonotrode operates in particular in the ultrasonic range with a frequency range from 20 kHz to 90 kHz, preferably from 20 kHz to 34 kHz. In particular, the sonotrode comprises steel, titanium, aluminum, and/or carbon. In particular, a sonotrode is a probe with for example a bar-shaped, tube-shaped, and/or hose-shaped embodiment. The sonotrode can be formed in one or more pieces. The sonotrode has in particular a diameter in a range of 0.5 mm to 4.5 mm, in particular of 0.8 mm to 3.8 mm.

An “acceleration tube” is in particular an elongated hollow body whose length has a greater dimension than its diameter. In its interior, the acceleration tube has a cavity in particular, in which a projectile can move freely in the longitudinal direction. Moreover, the acceleration tube in particular comprises a proximal end and a distal end which spatially define the maximum acceleration path, minus the projectile length. Distally and/or at its distal end portion, the acceleration tube is surrounded, in particular at least in part, by the horn and a bolt connected to or associated with the horn. In the case of a pneumatic force generation apparatus, the acceleration tube comprises at least one opening for the entrance and/or exit of a pressure medium, in particular compressed air. The acceleration tube comprises a metal in particular.

An “abutment element” in particular is in particular a desired endpoint of the movement of the projectile along the acceleration path within the cavity in the acceleration tube, at which the accelerated projectile impacts on the abutment element, is decelerated, and/or moved in the opposite direction. In particular, a distal-side abutment element is arranged at and/or in the distal end of the acceleration tube and/or within the cavity in a region of the distal portion of the acceleration tube. The distal-side abutment element transmits the shock of the projectile onto the sonotrode, in particular directly or indirectly. For example, the distal-side abutment element can be a proximal-side wall of the horn, a spring element, or a proximal-side wall of a holder for a spring element. In particular, a proximal-side abutment element is arranged at and/or in the proximal end of the acceleration tube or within the cavity in a proximal portion of the acceleration tube. For example, the proximal-side abutment element can be a wall of the housing, a receptacle for the acceleration tube, and/or a spring element.

A “projectile” is in particular a body which is freely movable along the acceleration path within the cavity in the acceleration tube. The projectile is movable in particular back and forth between the proximal-side abutment element and a distal-side abutment element within the cavity, arranged therebetween, in the acceleration tube. In principle, the projectile can have any shape. For example, the projectile can have the shape of a bolt or a ball. The projectile has in particular hard steel and/or weak magnetic properties. For the free mobility, the projectile has in particular a slightly smaller outer diameter than the diameter of the cavity in the acceleration tube. For example, the projectile can have an outer diameter of 8 mm, in particular 6 mm, or 4 mm.

In particular, the projectile can be moved back and/or forth along the acceleration path continuously or discontinuously by means of the force generation apparatus. Preferably, the projectile is moved back and forth in an intermittent and/or oscillating manner between the proximal-side abutment element and the distal-side abutment element.

In principle, a “force generation apparatus” can be any type of apparatus that applies a force to the projectile and thus causes a movement of the projectile. The force generation apparatus can be, for example, an apparatus which accelerates the projectile by means of a laser, a pressure medium, for example pneumatically by means of compressed air, by means of an electromagnetic field and/or by means of a mechanical apparatus. A pneumatic force generation apparatus can bring about a linear motion of the projectile in the cavity in the acceleration tube by means of a supply and/or removal of a pressure medium in particular. In particular, the pressure medium flows into the cavity in the acceleration tube through at least one proximal-side opening in the acceleration tube and presses and accelerates the projectile in the distal direction.

In particular, a “vibration excitation apparatus” is any apparatus for generating vibrations in the ultrasonic range. In particular, the vibration excitation apparatus comprises an ultrasonic transducer (also referred to as an ultrasonic converter) which converts a supplied AC voltage at a specific frequency into a mechanical vibration frequency; or the vibration excitation apparatus is formed by the ultrasonic transducer. In particular, the ultrasonic transducer is an electromechanical transducer that exploits the piezoelectric effect. As a result of applying the AC voltage generated by an ultrasonic generator, a mechanical vibration is generated on account of a deformation of the ultrasonic transducer. In particular, the ultrasonic transducer comprises a piezo element or a plurality of preferably stacked piezo elements. Preferably, the ultrasonic transducer comprises at least two piezo elements, with an electrical conductor, for example a copper plate, being arranged between the piezo elements. A distal-side piezo element of the ultrasonic transducer rests, especially directly, against a proximal wall of a horn. In particular, a counter bearing is arranged to the proximal side of the piezo element or the piezo elements. An intermediate plate can be arranged between the proximal end of the proximal-side piezo element and the distal end of the counter bearing. The piezo element, the piezo elements, the intermediate plate, and/or the counter bearing can be arranged in particular around a bolt, in particular a hollow bolt, which is arranged to the proximal side of the horn.

In particular, a “horn” is a component arranged between the ultrasonic transducer and/or a piezo element on the one hand and the sonotrode on the other hand. In particular, the horn serves to transfer the ultrasonic waves generated by the ultrasonic transducer to the sonotrode, and/or to transmit, to focus, and/or to align said ultrasonic waves. To this end, the horn may taper in a transfer direction and directly or indirectly transfer the ultrasonic waves to a probe head. In particular, an amplitude increase is obtained as a result of a cross-sectional reduction of the horn in the transfer direction. The horn can also be used for fastening the sonotrode. At the same time, the horn serves in particular together with a counter bearing and/or an intermediate plate for mechanically holding the piezo element or piezo elements on both sides. The horn terminates with a wall counter to the transfer direction, in particular on the proximal side. A bolt in particular is arranged to the proximal side of this wall. The bolt is preferably a hollow bolt. In particular, the horn and the bolt can be designed as two separate components. Preferably, the horn and the bolt are a one-piece component, with the horn portion corresponding to the conventional horn and merging into the hollow bolt portion with a smaller cross section, especially in graduated fashion, counter to the transfer direction, in particular in the proximal direction. At least one piezo element with an electrical contact and the counter bearing and/or additionally an intermediate plate between the proximal-side element and the distal side of the counter bearing arranged therebetween are arranged around the hollow bolt portion. In particular, the counter bearing is screwed on to the hollow bolt or the hollow bolt portion and as a result clamps at least one piezo element and/or the intermediate plate. The counter bearing can be designed as a screw nut. A proximal end portion of the hollow bolt portion and/or hollow bolt protrudes beyond the proximal end of the counter bearing, especially in the proximal direction.

In particular, a “vibration damping apparatus” (also referred to as “vibration absorber”) is any apparatus which quenches and/or damps vibrations caused by the shock excitation by means of a projectile and/or the vibration excitation by means of a vibration excitation apparatus and/or which at least partially decouples said vibrations from the housing. In particular, a vibration damping apparatus is a vibration damping component or an assembly comprising at least one mass and at least two spring elements, with at least one spring element contacting the mass of the vibration damping apparatus with its one end and contacting an inner surface of the housing with its other end. In particular, the mass is designed as a movable inertial mass and acts as an absorber mass, which is deflected from its rest position by the propagating vibrations and which has a retarding effect, while the at least two spring elements have a vibration damping embodiment. In this case, the weight of the mass and the spring constants of the spring elements, in particular, are designed such that these are matched to, and admit, a desired, specific frequency range of the lithotripsy device and/or an operational frequency of an ultrasonic vibration exciter of the vibration excitation apparatus. Together with the at least two spring elements, the mass forms a mass/spring system and/or a pendulum, the natural frequency of which is set to the vibration frequency or vibration frequencies to be eliminated. Preferably, the natural frequency of the vibration damping apparatus is set to an unwanted frequency to be eliminated and/or resonant frequency of a vibrating assembly, for example of the vibration excitation apparatus. At this frequency, the vibration absorber can carry out large vibration deflections, with the vibration absorber taking vibration energy from the vibrating assembly for its own vibration movement and converting said vibration energy into heat, and thereby extinguishing it, on account of friction. Consequently, the vibration damping apparatus prevents and/or reduces in particular the propagation and transmission of vibrations across components and/or assemblies within the housing, which could otherwise be transferred to the housing and/or the handpiece as vibrations.

In particular, a “spring element” is a component and/or a portion of the vibration damping apparatus that can be elastically deformed to a sufficient extent. In particular, the spring element comprises metal and/or plastic. In particular, a spring element can be a conventional spring, for example a coil spring, and hence a wire wound in a coil. In particular, the elastic deformation of the spring element is bending, torsion, extension, and/or compression. In addition to the vibration damping, the spring element serves in particular to hold the mass in its rest position and/or restore said mass to its rest position. In the process, a respective spring element is supported on the opposite end faces of the mass, preferably on both sides. In the case of a proximal-side arrangement within the housing in particular, the spring element is stretched in the case of an acceleration direction of the projectile in the distal direction and/or compressed in the case of an acceleration direction of the projectile in the proximal direction.

In particular, a “vibration compensation apparatus” (also referred to as “amplitude compensator”) is a component or an assembly comprising at least one mass and at least one spring element. In particular, the vibration compensation apparatus serves to vibrationally decouple the acceleration tube of the ballistic and/or pneumatic drive from the vibration excitation by means of the vibration excitation apparatus. In particular, the spring element is arranged on the distal side and the mass, as a rest mass, is arranged on the proximal side of the vibration compensation apparatus. The vibration compensation apparatus comprises in particular a continuous cavity in its mass and its spring element, through which the acceleration tube can be guided, with the result that the outer surface of the acceleration tube is surrounded by the vibration compensation apparatus.

As further components, the vibration compensation apparatus comprises in particular at least one connection element for connection to the bolt and/or the horn of the ultrasonic transducer and at least one sealing element, for example an O-ring. The sealing element acts as a damping element at the same time. The vibration compensation apparatus may also comprise a plurality of spring elements, for example arranged parallel to one another, and/or a plurality of masses. The spring element of the vibration compensation apparatus is in particular a thin-wall tube section which acts as a λ/4 mass-spring element in particular. The mass and/or the entire vibration compensation apparatus comprises aluminum and/or steel in particular. Preferably, the entire amplitude compensator comprises aluminum and/or an aluminum alloy. While the spring element of the vibration compensation apparatus vibrates during operation and thus has a damping effect, the mass remains at rest in particular on account of its significantly higher weight and precisely does not vibrate.

A “longitudinal center axis” is in particular the axis of the respective body or component which corresponds to the direction of its greatest extent and/or dimension. The longitudinal center axis can also be the axis of symmetry of the respective body and/or component.

In particular, a “longitudinal direction” is the direction of longest extent of a component and/or body. In particular, the longitudinal direction is the direction along the longitudinal center axis of the mass, sonotrode, and/or acceleration tube.

In a further embodiment of the holding device, the vibration damping apparatus comprises a third spring element and optionally further spring elements.

Stronger decoupling from the housing can be obtained by a third spring element and optionally further spring elements, by virtue of one of the two ends of the third and/or the respective further spring element contacting the mass and the other end contacting the inner surface of the housing. Likewise, the second end of the third and/or respective further spring element may also be in contact with an assembly, a component, and/or a holding receptacle within the housing rather than with the inner surface of the housing. By way of the third spring element and/or further spring elements, the vibration damping and/or decoupling can be damped and decoupled in a targeted manner by aligning and forming the respective spring element in different directions and/or with different intensities. In principle, it should be stressed that the two, three, or more spring elements may have the same design, but may also have different properties such as different lengths, spring constants, and/or dimensions.

In order to realize a compact arrangement of the vibration damping apparatus and an optimal use of the installation space available within the housing of the holding device, one assembly and/or a plurality of assemblies or all assemblies in the housing can each be formed as a mass of the vibration damping apparatus.

Hence, the vibration damping apparatus need not have a separate, own mass; instead, the components already present in the housing can each be used as a mass, which is in contact with at least one spring element. An optimal damping and decoupling of induced vibrations is obtained in a multiplicity of different spatial directions, especially in the case where a plurality of assemblies or all assemblies in the interior of the housing are each formed as a mass of the vibration damping apparatus, with the result that the entire outer surface of the handpiece has a low-vibration embodiment.

In a further embodiment of the holding device, the vibration excitation apparatus or a component of the vibration excitation apparatus is in the form of a mass of the vibration damping apparatus.

As a result, vibration absorption can be realized directly in the vibration excitation apparatus itself and can be set to damp undesirable vibrations. For example, the counter bearing of an ultrasonic transducer can be designed as a mass and directly connected to a spring element which is in contact with the inner surface of the housing or an assembly and/or a component within the housing at its other end.

In order to obtain vibration absorption in the longitudinal direction of the housing between the proximal-side abutment element and the distal-side abutment element, the acceleration tube is embodied as a mass of the vibration damping apparatus.

Hence, there can be vibration absorption by means of the acceleration tube as a mass over the entire length of the acceleration tube, with the spring elements being arrangeable at defined positions along the longitudinal direction of the acceleration tube, at which a targeted vibration damping should be set vis-à-vis the inner surface of the housing and/or any other component.

In a further embodiment of the holding device, the housing comprises a circuit board holder, the circuit board holder being in the form of a mass of the vibration damping apparatus.

Hence, the circuit board holder has the dual function of being a carrier element for electronic components within the holding device and a mass of the vibration damping apparatus.

In order to optimally integrate the vibration damping apparatus within the available installation space in the elongate housing, the two spring elements are each arranged on one side of the mass in the longitudinal direction and held by means of a holding unit.

As a result, the vibration damping apparatus can be inserted as a one-piece compact assembly into an available free space within the housing of the holding device.

A “holding unit” in particular is a holder for holding and/or fastening the vibration damping apparatus. The holding unit surrounds and/or carries the mass and/or the spring elements of the vibration damping apparatus at least in part. For example, the respective spring element can surround the holding unit or the holding unit is arranged around the respective spring element. The end of the spring element arranged opposite to that end on the mass can in particular press against a constituent part of the holding unit such that the holding unit forms a support bearing for the spring element opposite the mass. However, the holding unit may also have a free end face at the end of the spring element which contacts the inner surface of the housing and/or an assembly. Hence, the holding unit can for example be a tube in which the vibration damping apparatus is arranged, with an end of a spring element being arranged at each of the free tube ends, said spring element contacting an inner surface of the housing and/or an assembly. Likewise, the holding unit can be a piston, a rod or a rail, which for example is arranged on both sides of the mass and in each case is surrounded by a spring element.

In a further embodiment of the holding device, a first spring element is arranged to the proximal side of the vibration excitation apparatus and a second spring element is arranged to the distal side of the proximal end of the housing.

This achieves optimal damping and decoupling of vibrations caused by the recoil of the projectile following impact on the distal-side or proximal-side abutment element.

To achieve improved vibration decoupling between the ultrasonic unit and the acceleration tube, a vibration compensation apparatus can be arranged between the vibration excitation apparatus and the first spring element.

Consequently, vibrations of the handpiece are further reduced since there is an additional compensation of ultrasonic vibrations by means of the vibration compensation apparatus and undesirable excited vibrations on the acceleration tube, generated by the vibration excitation apparatus, are prevented or at least reduced, whereby the vibration excitation apparatus and the ballistic and/or pneumatic drive by means of the force generation apparatus are settable and operable independently of one another. Consequently, undesirable ultrasonic vibrations and/or transverse moments are initially compensated in the proximal direction by the vibration compensation apparatus and are subsequently absorbed and damped further by the vibration damping apparatus. Moreover, the horn, the bolt, and/or the ultrasonic transducer and also the vibration compensation apparatus can move within the housing and are decoupled from the inner surface of the housing by means of the vibration damping apparatus.

In a further embodiment, the mass is arranged concentrically around the acceleration tube, with each of the spring elements contacting an outer surface of the mass with their one end and contacting the inner surface of the housing with their other end.

The concentric arrangement of the mass as an absorber mass around the acceleration tube allows this mass to vibrate freely in three dimensions in the style of a pendulum around the acceleration tube on account of the spring elements arranged between the mass and the housing. As a result of the mass surrounding the acceleration tube concentrically, vibrations emanating from the acceleration tube or acting on the acceleration tube are optimally damped, without there being a mechanical contact between the acceleration tube and the mass. It is particularly advantageous if in this case the spring elements between the outer surface of the mass and the inner surface of the housing are distributed uniformly over the cross section of the concentrically arranged mass. In this case, the mass can be for example a circuit board holder which is arranged concentrically around the acceleration tube, especially in the proximal region of the acceleration tube. In addition to a radially compact design within the housing of the handpiece, radially uniformly aligned vibration absorption and damping is also obtained.

In order to further dampen the oscillations and hence the vibrations, the respective spring element and/or the holding unit has a shock absorber unit.

Consequently, the vibrations acting on the vibration damping apparatus and/or the movable mass decay faster and/or are reduced to a greater extent on account of the shock absorber unit.

In particular, a “shock absorber unit” is a component which makes the vibrations of the vibration damping apparatus and/or the movable mass of the vibration damping apparatus decay faster. In particular, the shock absorber unit converts the vibration energy into heat, whereby the vibrations are damped significantly and decay faster. A shock absorber unit can be a hydraulic damper with a hydraulic fluid or a friction damper.

In a further embodiment, the holding device comprises a horn distally and a bolt proximally of the horn, the horn and the bolt surrounding a distal portion of the acceleration tube, a counter bearing being arranged on the bolt proximally of the horn and at least one piezo element as a vibration exciter being arranged and mechanically coupled between the counter bearing and the horn, the horn comprising the distal-side abutment element and/or the horn being connectable to the distal-side abutment element and/or the sonotrode and the at least one piezo element being electrically connectable to an assignable ultrasonic generator, the vibration damping apparatus being arranged proximally on and/or of the horn, the bolt, and/or the counter bearing.

As a result of the proximal-side arrangement of the vibration damping apparatus directly in front of the ultrasonic transducer and/or a component of the ultrasonic transducer, undesirable vibrations in the proximal direction and/or transverse direction, excited by the ultrasonic transducer, can be damped and absorbed in a targeted manner, with the result that a further transmission in the proximal direction within the housing is prevented.

In a further aspect of the invention, the object is achieved by a lithotripsy device, in particular an intracorporeal lithotripsy device, for fragmenting calculi, wherein the lithotripsy device comprises a sonotrode and a holding device, and the holding device is a holding device as described above.

Consequently, a lithotripsy apparatus with a handpiece is provided, in which undesirable vibrations of the handpiece are largely prevented on account of the vibration damping apparatus in the handpiece, an efficient use of the installation space within the housing is made possible, and the targeted handling of the handpiece, and hence of the lithotripsy device, by the user is made possible, without the desired momentum transfer to the sonotrode by means of vibration excitation and shock excitation serving for fragmentation of calculi being restricted. The drawings, the description, and the claims contain numerous features in combination. It will be appreciated that the features mentioned above and the features yet to be explained below are applicable not only in the respectively specified combination but also in other combinations or on their own, without departing from the scope of the present invention.

### BRIEF DESCRIPTION OF THE DRAWINGS

The invention is explained hereinbelow using exemplary embodiments. In the drawing:

FIG. 1 shows a schematic three-dimensional representation of a lithotripsy device with a handpiece, a horn, and a sonotrode,

FIG. 2 shows a schematic illustration of the handpiece from FIG. 1 with the horn, an amplitude compensator, a vibration absorber, and an acceleration tube in full section,

FIG. 3 shows a very schematic illustration of the vibration absorber from FIG. 2, and

FIG. 4 shows a very schematic illustration of a handpiece and an alternative of a vibration absorber, with a mass arranged concentrically around an acceleration tube and three spring elements connected to a housing of the handpiece.

### DETAILED DESCRIPTION

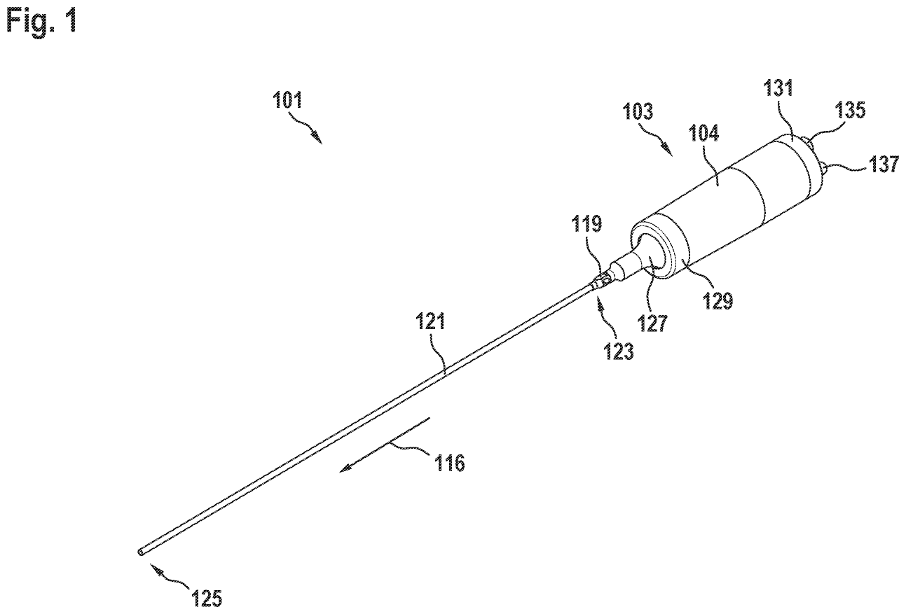

A lithotripsy device 101 comprises a handpiece 103 with a housing 104. At its proximal end, the housing 104 is terminated by a lid 131. An electrical connector 135 and a connection nozzle 137 for supplying compressed air are arranged on the proximal side of the lid 131. On the distal side, the housing 104 comprises a sleeve 129 which surrounds a horn 127. At its proximal end 123, a sonotrode 121 is screwed-in in the horn 127 by means of its sonotrode head 119. A distal end 125 of the sonotrode 121 opposite to the proximal end 123 serves for fragmenting calculi (FIG. 1).

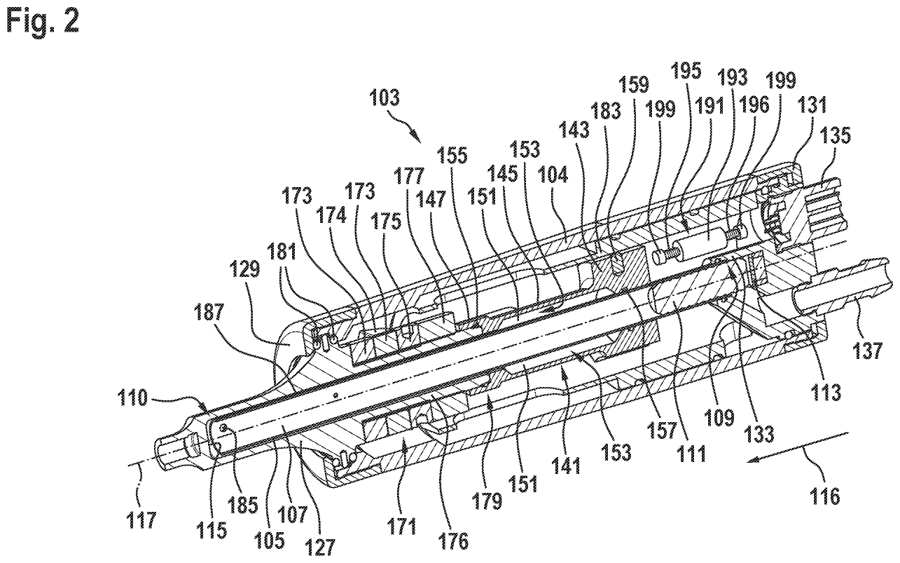

In a distal direction 116, the horn 127 has a tapering portion. To the proximal side of this tapering portion, the horn 127 merges into a hollow bolt 176 in one piece. The horn 127 is mounted in the housing 104 by means of two O-rings 181 at its largest cross section. An acceleration tube 105 which extends from its distal end 110 to its proximal end 109 along a longitudinal center axis 117 is arranged in the interior of the hollow horn 127 and the adjacent hollow bolt 176 (see FIG. 2). In the interior, the acceleration tube 105 comprises a cavity 107, in which a projectile 111 is movably arranged. The proximal end 109 of the acceleration tube 105 is held in a tube receptacle 133 within the housing 104. The cavity 107 of the acceleration tube 105 is fluid-connected to the connection nozzle 137. The projectile 111 is movable along the longitudinal center axis 117 in the cavity 107 of the acceleration tube 105, between a proximal-side abutment element 113 and a distal-side abutment element 115. The distal-side abutment element 115 is formed by a proximal-side wall of the horn 127.

Distally, an ultrasonic transducer 171 is arranged around the hollow bolt 176. The ultrasonic transducer 171 comprises two piezo elements 173 with an electrical conductor arranged therebetween and an electrical contact 174. The piezo elements 173 are clamped between the horn 127 and an intermediate plate 175 by means of a proximal-side counter bearing 177, with the intermediate plate 175 and the counter bearing 177 likewise surrounding the hollow bolt 176. An amplitude compensator 141 is arranged around the acceleration tube 105 at the proximal end 179 of the ultrasonic transducer 171 and in the central region of the housing 104. The amplitude compensator 141 is fabricated in one piece from aluminum and has a mass part 143 on the proximal side and a spring tube portion 145 on the distal side. The spring tube portion 145 has a connection portion 147 at its distal end. The connection portion 147 is screwed onto the proximal end of the hollow bolt 176 and sealed by means of an interior distal O-ring 155. On the inside, the amplitude compensator 141 has a cavity through which the acceleration tube 105 is guided. Additionally, the amplitude compensator 141 has a cutout 151 in its inner wall around the cavity, said cavity having been introduced into the spring tube portion 145 and a distal portion of the mass part 143 such that the amplitude compensator 141 has a compressed air reservoir 153 circumferentially around the acceleration tube 105 (FIG. 2).

The mass part 143 is sealed by way of a proximal O-ring 157 at the acceleration tube 105. As result of the amplitude compensator 141 only being sealed at the acceleration tube 105 to the proximal side by way of the proximal O-ring 157, the compressed air in the compressed air reservoir 153 formed by the cutout 151 can escape distally from the compressed air reservoir 153 through a compressed air channel 187 between the outer surface of the acceleration tube 105 and the inner surface of the distal portion of the amplitude compensator 141, hollow bolt 176, and horn 127 in the distal direction 116 and flow into the cavity 107 through an opening 185 at the distal end 110 of the acceleration tube 105 and/or through the open end face at the distal end 110 of the acceleration tube 105. Likewise, conversely, compressed air from the cavity 107 can be pressed into the compressed air channel 187 as intermediate space between the outer surface of the acceleration tube 105 and the inner surface of the horn 127 and hollow bolt 176 of the distal portion of the amplitude compensator 141 through the opening 185 and the open end face at the distal end 110 of the acceleration tube, pressed into the compressed air reservoir 153 counter to the distal direction 116 and collected in said compressed air reservoir when the projectile 111 is accelerated in the distal direction 116. In this case, the distal O-ring 155 between the connection portion 147 of the spring tube portion 145 and the proximal end of the hollow bolt 176 seals the compressed air channel 187 from the interior of the housing 104.

In the distal direction 116, a circuit board holder 183 surrounds the acceleration tube 105 from its proximal end 109 up to and including the amplitude compensator 141 and the counter holder 177. At its lateral surface, the mass part 143 of the amplitude compensator 141 is fastened in frictionally connected and interlocking fashion at points in flutes on the inner surface of the circuit board holder 183 by means of three radially uniformly spaced apart plastic pins 159. In turn, the circuit board holder 183 is in contact with the inner side of the housing 104 in radially circumferential fashion, with the result that the amplitude compensator 141 is indirectly connected to the housing 104 in the radial direction via the circuit board holder 183. As a result, the proximal end of the mass part 143 is precisely without a connection to the housing 104 and the lid 131 in the proximal direction.

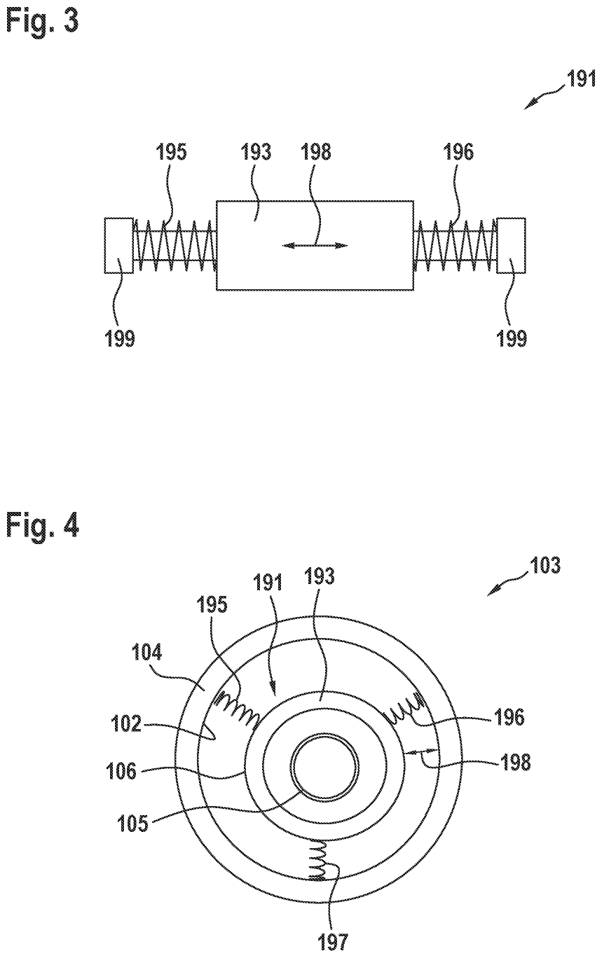

A vibration absorber 191 is arranged between the proximal-side wall of the mass part 143 of the amplitude compensator 141 and the distal-side wall of the tube receptacle 133. The vibration absorber 191 comprises a mass 193 as absorber mass and a first compression spring 195 and a second compression spring 196 on its opposite end faces. The first compression spring 195 and the second compression spring 196 are held by means of a holder 199, with the first compression spring 195 and the second compression spring 196 each being arranged around a piston of the holder 199 (see FIGS. 2 and 3). On account of the first compression spring 195 and the second compression spring 196 arranged on both sides, the mass 193 is movably mounted on both sides in an opposite damping direction 198.

The following operations are performed by means of the combined lithotripsy device 101 with a vibration excitation of the sonotrode 121 by means of the ultrasonic transducer 171 and a pneumatic drive for shock excitation of the sonotrode 121 by means of the projectile 111.

An ultrasound generator (not shown in the drawings) is used to apply a voltage to the ultrasonic transducer 171 by way of the electrical contact 174, whereby the piezo elements 173 are deformed within the ultrasonic transducer 171 and an ultrasonic vibration is induced as a result. The generated ultrasonic vibration is introduced into the sonotrode 121 on account of the conic portion of the horn 127, whereby the sonotrode 121 is excited to provide a vibration wave with a longitudinal vibration and in the transverse direction.

At the same time, a force generation apparatus (not shown) is used to press compressed air through the connection nozzle 137 into the cavity 107 at the proximal end 109 of the acceleration tube 105, whereby the projectile 111 moves along the longitudinal center axis 117 through the cavity 107 from the proximal end 109 as the initial state (see FIG. 2) in the distal direction 116 from the proximal-side abutment element 113 to the distal-side abutment element 115. As a result of the projectile 111 impacting on the distal-side abutment element 115, the shock from the projectile 111 is transferred to the sonotrode 121 via the distal end of the horn 127 and the sonotrode head 119. As a result of the acceleration of the projectile 111 in the distal direction 116, the air in the distal portion of the cavity 107 within the acceleration tube 105 is compressed and escapes counter to the distal direction 116 into the compressed air reservoir 153 of the amplitude compensator 141 through the opening 185 and the compressed air channel 187 between the outer surface of the acceleration tube 105 and the inner surface of the horn 127, the hollow bolt 176, and the distal portion of the amplitude compensator 141, with the compressed air being compressed in the compressed air reservoir 153. As a result of the impact of the projectile 111 on the distal-side abutment element 115, the projectile 111 is repulsed and, as a result of simultaneous closure of the supplied compressed air through the connection nozzle 137, the compressed air compressed in the compressed air reservoir 153 now flows in the distal direction 116 through the compressed air channel 187, the opening 185, and the open distal end face of the acceleration tube 105 into the cavity 107 and presses the projectile 111 back again toward the proximal end 109 of the acceleration tube 105, until the initial state (FIG. 2) has been reached again. This shock excitation of the sonotrode 121 as a result of the impact of the projectile 111 on the distal-side abutment element 115 is repeated on a regular basis.

The ultrasonic vibrations generated by means of the ultrasonic transducer 171 have a frequency of approximately 27 kHz, to which the amplitude compensator 141 is matched exactly. As a result of the amplitude compensator 141 having a λ/4 geometry, which corresponds to the resonant frequency of the ultrasonic transducer 171, the ultrasonic transducer 171 is not detuned by the amplitude compensator 141. On account of the λ/4 geometry of the amplitude compensator 141, the vibration wave generated by means of the ultrasonic transducer 171 impacts with its amplitude maximum at a quarter wavelength on the spring tube portion 145, which vibrates on account of its elastic properties and absorbs and damps this amplitude, with the result that the mass part 143 as rest mass moves only negligibly, if at all, on account of the small residual ultrasound amplitude. In this case, the radially circumferentially arranged plastic pins 159 for a punctiform mount and the proximal O-ring 157 have an additional damping action, with the result that an abrasion, other types of damage, and heating in the mass part 143 are negligible. Moreover, metallic rattling at the circuit board holder 183 is prevented by the punctiform mount by means of the plastic pins 159, by means of which possibly present transverse moments are dissipated radially to the outside.

As a result of the amplitude compensator 141, at its lateral surface, being mounted radially to the outside on the circuit board holder 183 by means of the plastic pins 159 and the proximal end of the mass part 143 being free in the proximal direction and precisely not connected to the housing 104 and the lid 131, the acceleration tube 105 is optimally vibration-decoupled from the ultrasonic transducer 171 by means of the amplitude compensator 141, with the result that the pneumatic drive of the projectile 111 in the acceleration tube 105 is operable independently of the ultrasonic vibration generated by means of the ultrasonic transducer 171 and both the drives are settable independently of one another.

Possible residual vibrations which, generated by the ultrasonic transducer 171, occur to the proximal side of the amplitude compensator 141 despite the amplitude compensator 141 and which can bring about undesirable vibrations of the housing 104 are absorbed by means of the vibration absorber 191 on account of its mass 193 and damped by means of the first compression spring 195 and the second compression spring 196. In this case, the vibration absorber 191 is matched to the frequency of the residual vibrations to be eliminated, while the desired frequency of 27 kHz of the ultrasonic transducer 171 is not impaired. Excited by the residual vibrations, the movable mass 193 implements a large deflection movement, to alternating sides in the damping direction 198, with vibration energy being withdrawn for this deflection and being converted into heat on account of friction by means of the first compression spring 195 and the second compression spring 196, with the result that the residual vibrations are reduced.

In the case of the above-described acceleration of the projectile 111 in the distal direction 119, the pressure on the projectile 111 simultaneously also acts on the housing 104 of the grip 103, whereby the housing 104 withdraws in the opposite direction and the first compression spring 195 and the second compression spring 196 of the vibration absorber 191 are each stretched on both sides in the opposite damping direction 198. Following the repulsion of the projectile 111 at the distal-side abutment element 115, the projectile 111 moves oppositely in the proximal direction and the housing 104 accordingly moves oppositely in the distal direction 116, with the first compression spring 195 and the second compression spring 196 of the vibration absorber 191 being compressed and moving toward one another. The stretch or compression of the first compression spring 195 and the second compression spring 196 firstly equalizes and compensates the opposing movement between the projectile 111 and the housing 104 when the projectile is accelerated in the distal direction 116 or conversely in the proximal direction, and secondly vibrations within the housing 104, which arise due to the recoil of the projectile 111 at the distal abutment element 115 or the proximal abutment element 113 in each case, are absorbed by means of the mass 193 and damped by means of the first compression spring 195 and the second compression spring 196.

Consequently, when the sonotrode 121 is used for direct fragmentation of calculi, both the vibration excitation of the sonotrode 121 by means of the ultrasonic transducer 171 and the shock excitation by the projectile 111 are usable with an effective high fragmentation performance, with vibrations induced by the vibration excitation and shock excitation of the sonotrode 121 being largely reduced by means of the vibration absorber 191. As a result, the user can guide the grip 103 smoothly and in a targeted manner and hence align the distal end 125 of the sonotrode 121 positionally accurately at the calculus to be fragmented.

In an alternative of the lithotripsy device 101 not shown here, the grip 103 does not have an amplitude compensator 141 in its housing 104; instead, the vibration absorber 191 is arranged with its first compression spring 195 directly against the proximal wall of the counter bearing 177. In this case, the vibration absorber 191 is matched directly to undesirable vibrations generated by the ultrasonic transducer 171. Otherwise, the lithotripsy device 101 and the vibration absorber 191 are operated as described above.

In an alternative of the vibration absorber 191 shown in FIG. 4, which shows a grip 103 in a cross-sectional illustration, the mass 193 of the vibration absorber 191 has a tubular form and is arranged concentrically around the acceleration tube 105 without contact. Arranged on the outer surface 106 of the mass 193 with a respective end are a first compression spring 195, a second compression spring 196, and a third compression spring 197, which are each in contact with an inner surface 102 of the housing 104 with their opposite end. The first compression spring 195, the second compression spring 196, and the third compression spring 197 are radially movable in a damping direction 198, and hence stretchable and compressible, both in the direction toward the inner surface 102 of the housing 104 and in the direction toward the outer surface 106 of the mass 193. During operation in an above-described lithotripsy device 101, the mass 193 vibrates three dimensionally and radially around the acceleration tube 105, without coming into contact with the latter, on account of the first compression spring 195, the second compression spring 196, and the third compression spring 197, whereby optimal vibration absorption and damping with respect to the surrounding housing 104 is obtained in radially circumferential fashion.

Consequently, a vibration absorber 191 is provided which, depending on the vibrations occurring in the housing 104 and the vibrations to be reduced, is able to be formed in a targeted manner with a mass 193 and spring elements 195, 196, 197 and is arrangeable within the housing 104.

The drawings, the description, and the claims contain numerous features in combination. It will be appreciated that the aforementioned features are applicable not only in the respectively specified combination but also in other combinations or on their own, without departing from the scope of the present invention. The invention relates to a holding device for a lithotripsy device for fragmenting calculi, the holding device comprising a housing for accommodating assemblies and/or components, and a sonotrode being connectable to the distal end of the housing, arranged within the housing there being an acceleration tube with a longitudinal center axis, a cavity, and with a movable projectile within the cavity for shock excitation of the sonotrode, a proximal-side abutment element arranged at the proximal end, and a distal-side abutment element arranged at the distal end of the acceleration tube, and the holding device being assignable a force generation apparatus for generating a force for moving the projectile back and/or forth, and a vibration excitation apparatus for exciting vibrations of the sonotrode and a vibration damping apparatus being arranged in the housing, wherein the vibration damping apparatus comprises at least one mass and at least two spring elements with two ends each, the at least two spring elements contacting the mass with their respective one end and at least one spring element, with its second end, contacting an inner surface of the housing. The invention also relates to a lithotripsy device.

### LIST OF REFERENCE SIGNS

101 Lithotripsy device
103 Grip
102 Inner surface
104 Housing
105 Acceleration tube
106 Outer surface
107 Cavity
109 Proximal end
110 Distal end
111 Projectile
113 Proximal-side abutment element
115 Distal-side abutment element
116 Distal direction
117 Longitudinal center axis
119 Sonotrode head
121 Sonotrode
123 Proximal end of the sonotrode
125 Distal end of the sonotrode
127 Horn
129 Sleeve
131 Lid
133 Tube receptacle
135 Electrical connection
137 Connection nozzle
141 Amplitude compensator
143 Mass part
145 Spring tube portion
147 Connection portion
151 Cutout
153 Compressed air reservoir
155 Distal O-ring
157 Proximal O-ring
159 Plastic pin
171 Ultrasonic transducer
173 Piezo element
174 Electrical contact
175 Intermediate plate
176 Hollow bolt
177 Counter bearing
179 Proximal end of the ultrasonic transducer
181 O-ring
183 Circuit board holder
187 Compressed air channel
191 Vibration absorber
193 Mass
195 First compression spring
196 Second compression spring
197 Third compression spring
198 Damping direction
199 Holder

1. A holding device configured for a lithotripsy device and configured for fragmenting calculi, the holding device comprising:
a housing configured to accommodate assemblies and/or components, and the housing comprising a distal end and a proximal end and a sonotrode being connectable to the distal end, arranged within the housing there being, as an assembly, an acceleration tube with a longitudinal center axis, a cavity, a proximal end, and a distal end, and with a movable projectile within the cavity configured for shock excitation of the sonotrode, a proximal-side abutment element arranged at the proximal end, and a distal-side abutment element arranged at the distal end of the acceleration tube, and
the holding device being assignable a force generation apparatus configured to generate a force to move the projectile back and/or forth between the proximal-side abutment element and the distal-side abutment element, and, as an assembly, a vibration excitation apparatus configured to excite vibrations of the sonotrode and a vibration damping apparatus being arranged in the housing,
wherein the vibration damping apparatus comprises at least one mass and at least two spring elements with two ends each, the at least two spring elements contacting the mass with their respective one end and at least one spring element, with its second end, contacting an inner surface of the housing.

2. The holding device as claimed in claim 1, wherein the vibration damping apparatus comprises one or more of: a third spring element and further spring elements.

3. The holding device as claimed in claim 1, wherein an assembly, a plurality of assemblies, and/or all assemblies in the housing are each a respective mass of the vibration damping apparatus.

4. The holding device as claimed in claim 1, wherein the vibration excitation apparatus or a component of the vibration excitation apparatus is a mass of the vibration damping apparatus.

5. The holding device as claimed in claim 1, wherein the acceleration tube is a mass of the vibration damping apparatus.

6. The holding device as claimed in claim 1, wherein the housing comprises a circuit board holder, the circuit board holder being a mass of the vibration damping apparatus.

7. The holding device as claimed in claim 1, wherein the two spring elements are each arranged on one side of the mass in the longitudinal direction and held by a holding unit.

8. The holding device as claimed in claim 7, wherein a first spring element is arranged to the proximal side of the vibration excitation apparatus and a second spring element is arranged to the distal side of the proximal end of the housing.

9. The holding device as claimed in claim 8, wherein a vibration compensation apparatus is arranged between the vibration excitation apparatus and the first spring element.

10. The holding device as claimed in claim 1, wherein the mass is arranged concentrically around the acceleration tube, with each of the spring elements contacting an outer surface of the mass with their one end and contacting the inner surface of the housing with their other end.

11. The holding device as claimed in claim 1, wherein the respective spring element and/or the holding unit comprises a shock absorber unit.

12. The holding device as claimed in claim 1, wherein the holding device comprises a horn distally and a bolt proximally of the horn, the horn and the bolt surrounding a distal portion of the acceleration tube, a counter bearing being arranged on the bolt proximally of the horn, and at least one piezo element as a vibration exciter being arranged and mechanically coupled between the counter bearing and the horn,
the horn comprising the distal-side abutment element and/or the horn, being connectable to the distal-side abutment element and/or the sonotrode and the at least one piezo element being electrically connectable to an assignable ultrasonic generator, the vibration damping apparatus being arranged proximally on and/or of the horn, the bolt, and/or the counter bearing.

13. A lithotripsy device configured to fragment calculi, the lithotripsy device comprising a sonotrode and a holding device, wherein the holding device is a holding device as claimed in claim 1.
